(19) United States
(12) Patent Application Publication
(10) Pub. No.: US 2017/0095367 A1
(43) Pub. Date: Apr. 6, 2017

(54) BODY TEMPERATURE REGULATING DEVICE AND METHOD

(71) Applicant: Mario LINI, San Giorgio Su Legnano (MI) (IT)

(72) Inventor: Mario LINI, San Giorgio Su Legnano (MI) (IT)

(21) Appl. No.: 15/126,518

(22) PCT Filed: Apr. 17, 2014
(86) PCT No.: PCT/IT2014/000112

§ 371 Date: Sep. 15, 2016

(30) Foreign Application Priority Data
Mar. 28, 2014 (IT) MI2014A00549

(51) Int. Cl. A61F 7/00

(52) U.S. Cl. CPC A61F 7/007; A61F 2007/0075; A61F 2007/0095; A61F 2007/0093; A61F 2007/0035; A61F 2007/0078

(57) ABSTRACT

A device for regulating body temperature includes a case with an opening in the base, a plate made of highly thermally conductive material with a complementary shaped protrusion at the opening and that is inserted therein, at least a Peltier cell, an electronic circuit board for managing the functions of the device, a heat sink, a display, a battery to power the electronic circuit board, the Peltier cell and the display, a temperature sensor to measure the temperature of the skin at the surface of the case and of the plate that is in contact with the skin, a strap which is secured to said concave case, a lid for closing said case, and at least one command button operatively connected to the electronic circuit board.

### TECHNICAL FIELD

The device referred to in this invention is in the field of human body thermoregulation apparatuses and, more particularly, relates to a device and a method for regulating body temperature.

As is known in the art, the term thermoregulation means the ability of an organism to contain its body temperature within a given range, even in the presence of an ambient temperature that differs considerably from the temperature of the body itself Adaptation to different climatic conditions is achieved by means of physiological mechanisms on the part of the liver, brain and heart, and by contraction of the skeletal muscles. Ignoring the processes of heat generation and dissipation based on the human body's ability to adapt to different temperatures, as regards instead the exchange of heat by the body with the surrounding environment it is known that this is implemented by means of the processes of convection, conduction, radiation and evaporation.

For example, when the need arises to reduce the temperature of the body one solution is to employ a means that will allow air to move over the skin thus facilitating evaporation which then results in a reduction in body temperature. Rather than only relying on transpiration this process can be facilitated by moistening the skin with a liquid and thereby also making use of heat transfer by conduction.

Another alternative for transferring heat to and from the human body consists in the use of thermoelectric devices such as the “Peltier cell” for example. This consists in a thermoelectric device made up of a series of “Peltier” effect junctions that result in a solid state heat pump which is then capable of generating a temperature gradient when placed in contact with the skin.

### PRIOR ART

There already exist body thermoregulation devices known in the art that employ a Peltier cell as a means to control body temperature, one of these is described in the Japanese Patent No. JP2004263325.

This device consists in an item of clothing equipped with a series of Peltier cells which are encapsulated within the fabric with which the garment is made. The cells are moreover powered by a battery and controlled by a temperature control unit. This device however has a reduced energy efficiency problem which is due to the inability to efficiently adhere the Peltier cells to the surface of the skin for transferring heat to the body of the user.

A further body thermoregulation device is described in the Japanese Patent No. JP2008031581. This device relates to body thermoregulation equipment which also takes the form of a garment fitted with a Peltier cell based heating and cooling system. The device described together with its relative thermoregulation equipment features large dimensions and is not practical in use because of poor adhesion between the “Peltier” cells and the skin and so therefore this equipment too does not present high energy transmission efficiency to and from the user's body.

A further body thermoregulation device is described in the Japanese Patent No. JP2012161446. This body thermoregulation equipment consists in a collar equipped with a system for heat exchange by means of the use of “Peltier” cells together with a fluid. This equipment too however has drawbacks including its large dimensions, in particular with regard to the device's location around the neck of the user which would severely restrict its use in certain contexts.

Other body thermoregulation devices are described in the Patent Nos. JP2010082427 and JP2002512081 both of which include the same drawbacks as the devices mentioned above.

Summarising then, the body thermoregulation devices known in the art have the following drawbacks: large dimensions which severely limit their use, reduced energy efficiency due to poor adhesion between the Peltier cells and the skin, and also none of the known equipment for body thermoregulation is equipped with a real time body temperature display element that allows the user to adjust the desired thermal functionality of the equipment in question.

### DESCRIPTION OF THE INVENTION

The objective of the present invention is to provide an equipment that solves the drawbacks associated with those body thermoregulation devices known in the art and, in detail, to provide a body thermoregulation device that features reduced dimensions, high heat transmission energy efficiency and that is applied to the user's wrist in order to transmit or extract heat.

Another objective of the present invention is to provide a body thermoregulation device equipped with a body temperature display element that allows the user to adjust the temperature of the body following the display and evaluation of the body's instantaneous temperature.

A further objective of the present invention is to allow for relaxation of the skin by reducing stress and increasing the feeling of wellness thus bringing the energy fields into balance.

### BRIEF DESCRIPTION OF THE DRAWINGS

These and further objectives are achieved by the body thermoregulation device, described below in a preferred embodiment which is not limited by further developments to the patent, with the aid of the attached drawings which illustrate, respectively:

FIG. 1 an exploded view of the body thermoregulation device referred to in the present invention;

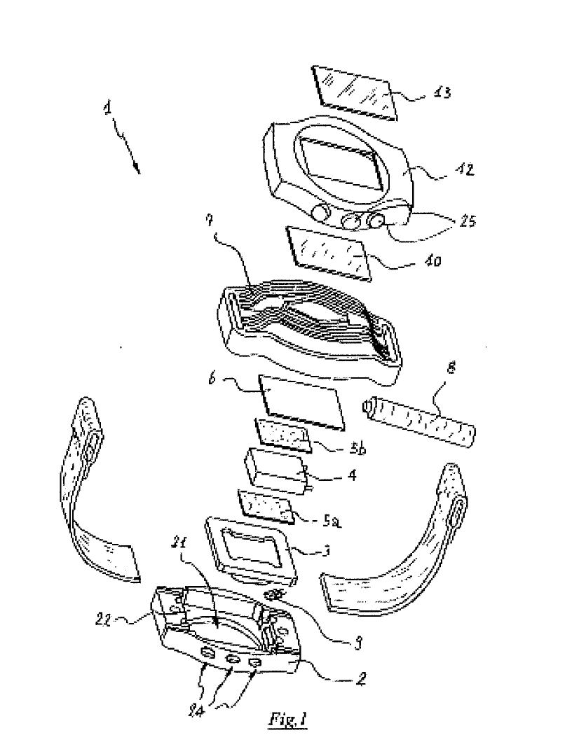

FIG. 2 a sectional view of the body thermoregulation device referred to in the present invention;

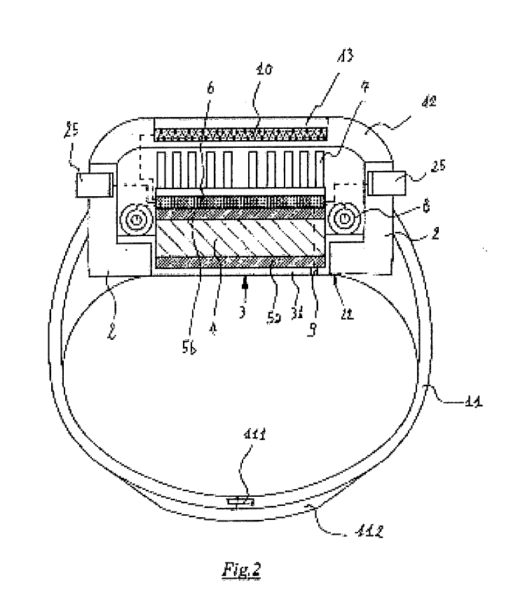

FIG. 3 a view of the body thermoregulation device display.

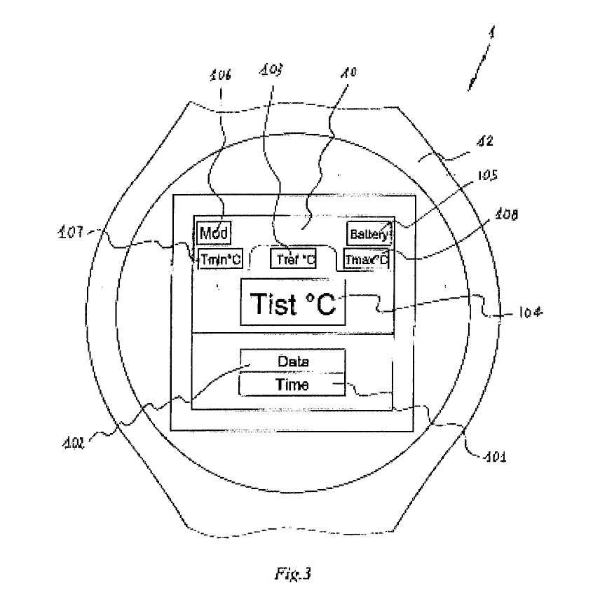

### DETAILED DESCRIPTION OF THE PREFERRED EMBODIMENTS

With reference to the attached figures, the device representing the present invention and relating to a body thermoregulation device 1 consists of a case 2 within which are inserted in sequence, starting from the base 22, a plate made of highly thermally conductive material 3 which is equipped with a temperature sensor 9, a thermal conduction interface 5a, a Peltier cell 4, a second thermal conduction interface 5b, a heat sink 7, a display 10 which is mounted on a lid 12 which allows for hermetic closure of the case 2 and which is equipped with a glass window 13 designed to protect the display 10.

Again, within the case 2, there are to be found an electronic circuit board 6 and one or more batteries 8 for supplying power to the device 1; moreover, said case 2 is held in position by a strap 11 in such a way that the device 1 encircles the wrist W of the user. Finally, the case 2 and lid 12 feature holes 24 for locating control buttons 25 that are operatively connected to the electronic circuit board 6 in order to interact with the latter for the setting or modification of the device operating parameters.

With reference to the attached figures, the case 2 may furthermore present an opening 21 at its base whose shape corresponds to that of the bottom projection 31 which is positioned on the face 3a of the plate 3, said plate being made of highly thermally conductive material. The thickness of said bottom projection 31 coincides with the thickness of the base 22 of the case 2.

The plate 3 made of highly thermally conductive material transmits the heat from the Peltier cell 4 to the skin, or vice versa. To this aim, the plate 3 is made of highly thermally conductivity material, such as aluminium for example, and is in contact with the skin, by which means heat exchange occurs whenever the device 1 is worn.

In the absence of the opening 21, the base 22 of the case 2 may be closed and placed in contact with a face 4a of the Peltier cell 4.

In order to increase heat exchange, thermal conduction interfaces 5a and 5b may be present which serve to enclose the Peltier cell 4; in particular the interfaces 5a and 5b are designed to increase the thermal exchange between the Peltier cell 4 and the plate 3, made of highly thermally conductive material, and between the Peltier cell 4 and the heat sink 7, the latter also being in contact with the Peltier cell 4.

As previously mentioned, the Peltier cell 4 is a thermoelectric device comprising a plurality of “Peltier” effect junctions which together constitute a solid state heat pump, wherein one surface absorbs heat while the other produces it and the direction in which the heat is transferred depends on the direction of the continuous current applied across the cell itself.

The electronic circuit board 6 comprises all of the components required in order to correctly manage the operation of the device. Therefore it is operatively connected to the display 10, temperature sensor 9, Peltier cell 4, control buttons 25 and is powered by the battery 8. The electronic circuit board performs the following tasks: by means of the sensor 9 it measures the skin temperature and stores the measured value in memory, it performs algebraic and comparison operations, it activates the Peltier cell 4 resulting in heat exchange, and sends temperature and functional information to the display 10. The electronic circuit board 6 is also capable of interpreting control signals derived from the buttons 25.

The display 10 reveals all of the information relating to the operation of the device including the temperature measured, information about the current date and time, as per a common digital clock, and allows for programming of the thermal functions. More specifically, the display 10 reveals the following information: time 101, date 102, reference skin temperature 103, instantaneous skin temperature 104, battery charge level 105, thermal function state (on/off) and thermal function mode (heating/cooling) 106 and the minimum 107 and maximum 108 temperatures. Furthermore, the display 10 is programmed in such a way as to vary its colour as a function of the reference temperature and as a function of the thermal function mode.

The heat sink 7 allows some of the heat produced by the Peltier cell 4 to be dissipated toward the outside of the device 1.

Once placed in position on the wrist and activated, for a predetermined period of time “T”, lasting 10 hours for example, the body thermoregulation device 1 begins a preliminary body temperature acquisition phase during which period the skin temperature is measured every “N” intervals over a predetermined duration of time “T1”, such as for example on the hour during the 10 hours of the learning phase.

The reference temperature measurement phase is managed by the electronic circuit board 6 which, to this aim, stores in memory the values measured by the temperature sensor 9, said sensor being in contact with the skin.

At the end of the temperature acquisition phase, the electronic circuit board 6 calculates the average temperature (also referred to as the reference temperature “TR”) whose value 103 is then revealed on the display 10.

The reference temperature TR, which is the average of the temperatures measured during the period T of the acquisition phase, is constantly updated by further temperature measurements occurring at a predetermined time interval “T2”, for example after every hour from the last reference temperature TR measurement event. Therefore, in the example above, the new reference temperature TR will be the result of the average of the temperatures measured over the last 10 hours excluding the most chronologically distant measurement and including instead the most recent measurement. The device 1 thus constantly updates the reference temperature TR during use.

Other then when the device is placed in position on the wrist and activated, the reference temperature TR acquisition phase can also be manually initiated via a reset button.

During said reference temperature TR acquisition phase, in order to allow for stabilisation of the temperature at the plate 3 made of highly thermally conductive material of the device 1 with respect to that of the skin, the Peltier cell 4 thermal heat transfer function is deactivated. Moreover, at this stage, an icon 106 appears on the display 10, for example an hourglass, indicating that the device 1 is occupied with the reference temperature acquisition phase.

When the device is not worn, the reference temperature and minimum/maximum temperature calculation process carried out by the electronic circuit board 6 is interrupted; the device will start making measurements again when it is once more in position on the wrist. To this aim there is a sensor 111 on the strap 11 that the electronic circuit board 6 uses to detect whether or not the device 1 is being worn.

Whenever the strap 11 is closed, a reactivation request or else a thermal function request will appear on the display 10.

The strap 11 can furthermore take the form of a strap with a Velcro 112, buckle or deployante buckle closure element.

Skin temperature measurements can also be performed manually by means of a dedicated control button 25.

The body thermoregulation device 1 features two operating modes: a first mode that aims to maintain the body temperature close to the reference temperature TR, and a second mode that can only be activated when the first mode is deactivated and that causes the Peltier cell to be activated cyclically over a given time interval “T4”. Said cyclic activation can be programmed to either only heat or only cool the surface of the skin which is in contact with the bottom of the plate 3.

An “automatic” mode is also included wherein the device 1 alternately heats and then cools for a predetermined period of time.

In order to avoid false temperature measurements, when the Peltier cell 4 is active and simultaneously a reference temperature measurement is being performed at the end of the temperature range “T1”, then before the temperature measurement is carried out by means of the temperature sensor 9 the electronic circuit board 6 first disables the Peltier cell 4.

The device also features a button 25 which allows the user to reset the reference temperature “TR” at any time thereby restarting the reference temperature learning phase.

As mentioned previously, the device features two operating modes, in the first case after the previously set time interval “T3” has elapsed the electronic circuit board 6, by means of the temperature sensor 9, performs an instantaneous temperature measurement “TS”, whose resulting value 104 also appears on the display 10, and compares it with the reference temperature “TR”. If the difference is greater or less than a given hysteresis value (e.g. a default value of 0.5° C. that can be manually set with increments of 0.5° C. to 2° C.) the electronic circuit board 6 will activate the Peltier cell 4 as a function of heating or cooling so as to rebalance the body temperature.

The Peltier cell 4 thermal functions will only be deactivated when the measured temperature TS is re-established, or else returns to within the hysteresis value set for the reference temperature TS=TR.

Alternatively to the first thermal operating mode, the electronic circuit board 6 can control the device 1 in such a way that after a predetermined number of hours the Peltier cell 4 is activated for a given period of time “T4”. In this operating mode the device firstly heats for a given duration and then, after a time interval, the device cools for the same duration.

Alternatively, the device can be set for a single heating or cooling cycle that will last for a manually preset duration.

In this operating mode the new temperature to be reached and maintained becomes the preset temperature “TP” which may be n-degrees higher or lower, up to the maximum difference in temperature guaranteed by the device. To this end the preset temperature “TP”, as chosen by the user, is set by means of the control buttons 25 that the device 1 features.

Following this logic the thermal functionality automatically turns off after a given interval at which point the temperature to be maintained returns to that of the reference temperature TR.

A schematic example is given of the operating phases of the device together with the defined time intervals:
0) The reference temperature TR learning phase has a total duration T of 10 hours during which the temperature is measured at every interval T1 (i.e. on the hour), at which point in time the Peltier cell 4 is turned off so as not to influence the measurement itself.
1) Once the reference temperature TR has been calculated, then at each successive interval T2, after 2 seconds for example, the instantaneous temperature TS is measured and used to maintain the reference temperature TR (TR=TS) by means of appropriately activating the Peltier cell 4.
2) After a predetermined time interval, for example 55 minutes, the Peltier cell is then deactivated.
3) After a further fixed time interval, for example 5 minutes, a temperature measurement is made in order to update the average calculated over the preceding 10 hours, i.e. the reference temperature TR, at which point the display is updated with the most recently measured temperature TS 104 and with the new reference temperature TR 103.
4) If for example two hours have passed since the last activation of the Peltier cell 4, and the device 1 thermal operating mode is set such that the Peltier cell 4 is cyclically activated after a set number of hours for a predetermined period of time, then the device either continues from phase five or else recommences from phase one.
5) The Peltier cell 4 is activated in order to either heat or cool with a maximum temperature difference of 3 degrees thus returning the set temperature to the reference temperature TR for example.
6) At each successive interval T1, after 2 seconds for example, the instantaneous temperature TS is measured and used to maintain the reference temperature TR by means of appropriately activating the Peltier cell 4.
7) After a predetermined time interval, for example 55 minutes, the Peltier cell is then deactivated.
8) After a further fixed time interval, for example 5 minutes, a temperature measurement is made in order to update the average calculated over the preceding 10 hours, i.e. the reference temperature TR, at which point the display is updated with the most recently measured temperature TS 104 and with the new reference temperature TR 103 and then the device recommences from phase four.

The time interval durations T, T1 and T2, the number of intervals N and the temperature TR are illustrative and may be different than those described and set by the user.

1. A device for regulating body temperature (1) comprising:
a case (2) having an opening (21) in the base (22);
a plate made of highly thermally conductive material (3) that features a complementary shaped protrusion (31) at the opening (21) and that is inserted therein;
at least one Peltier cell (4);
an electronic circuit board (6) for managing the functions of the device (1);
a heat sink (7);
a display (10);
a battery (8) to power said electronic circuit board (6), said Peltier cell (4) and said display (10);
a temperature sensor (9) designed to measure the temperature of the skin at the surface of the case (2) and of the plate (3) which is in contact with the same skin;
a strap (11), which is secured to said concave case (2);
a lid (12) for closing the case (2);
at least one command button (25) which is operatively connected to the electronic circuit board (6);
characterised in that within the case (2), starting from the base (22), a plate made of highly thermally conductive material (3), a temperature sensor (9), a Peltier cell (4), a heat sink (7), an electronic circuit board (6), a display (10) and a lid for the case (12) are sequentially located, said Peltier cell being activated whenever the skin temperature as measured by said temperature sensor (9) differs from the reference temperature value TR stored within the memory on the circuit board (6).

2. A device for regulating body temperature (1) according to claim 1, characterised in that the reference temperature TR stored within the memory on the electronic circuit board (6), is determined by the average temperatures of the skin, which are measured over an overall time interval T by means of the temperature sensor (9) which is operatively connected to said electronic circuit board (6), consisting of a predetermined temperature “TP” that is set by means of the at least one control button (25) , which is directly activated by the user of the device (1).

3. A device for regulating body temperature according to the preceding claims, characterised in that the display (10), which is operatively connected to the electronic circuit board (6), displays the following information: time (101), date (102), reference skin temperature TR (103), instantaneous skin temperature (104), battery charge level (105), thermal function on/off state and thermal heating or cooling operating mode (106), minimum temperature measured (107), maximum temperature measured (108).

4. A device according to claim 3 characterised in that the display (10) changes colour as a function of the measured temperatures.

5. A device according to the preceding claims, characterised in that at least a command button (25) is operatively connected to the electronic circuit board (6) in order to interact with the latter such that the operating parameters may be set or changed.

6. A device according to the preceding claims, characterised in that between the plate made of highly thermally conductive material (3) and the Peltier cell (4) a thermal conduction interface (5a) is interposed, and between the same Peltier cell (4) and the heat sink (7) a second thermal conduction interface (5b) is interposed.

7. A device according to claims 1, 2, 3 and 4 characterised in that one side of the Peltier cell (4) is in contact with a heat sink (7).

8. A device according to the preceding claims, characterised in that the device itself is maintained in contact with the skin of the user's wrist by means of a strap (11).

9. A device according to the preceding claims, characterised in that the strap (11) is equipped with a sensor (111) which is suitable for communicating the closure of said strap (11) to the electronic circuit board (6).

10. A body thermoregulation method by means of the device (1) which is characterised by the following steps:
powering on the device (1) by means of the at least one command button (25);
acquisition and storage in memory, on the electronic circuit board (6), of the temperature of the skin which is in contact with the case (2) and of the plate of highly thermally conductive material (3), by means of the temperature sensor (9), after N intervals of a predetermined time interval “T1” measured during the overall time interval “T”;
transmission of measured temperatures to the electronic circuit board (6);
calculation of the average temperature, as derived from the values measured during the overall time interval T which will then constitute the value for the reference temperature TR, by the electronic card (6);
acquisition of the instantaneous temperature “TS” by means of the temperature sensor (9), and comparison between the reference temperature “TR” and the “TS” at each previously set time interval “T3”;
activation of the Peltier cell (4) whenever the instantaneous temperature “TS” differs from the reference temperature “TR”, namely the cooling of the Peltier cell when the instantaneous temperature “TS” is higher than the reference temperature “TR”, or the heating of the Peltier cell (4) when the instantaneous temperature TS is lower than the reference temperature “TR”;
disactivation of the Peltier cell (4) during those conditions wherein the instantaneous temperature “TS” corresponds to the reference temperature “TR;
updating of the reference temperature “TR” by detecting the skin temperature by means of the temperature sensor (9) at the end of each new interval “T2”, following the period T;
recalculation of the new reference temperature “TR” by the electronic circuit board (6), consisting of the average of the temperatures measured over the last N−1 measurements, excluding the most chronologically distant measurement and in its place including the most recent measurement.
